(19) United States
(12) Patent Application Publication
(10) Pub. No.: US 2006/0199168 A1
(43) Pub. Date: Sep. 7, 2006

(54) Cell recovery

(76) Inventors: Ivan Nicholas Wills, Tempe (AU); David Robert James Monaghan, Botany (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/516,206

(22) PCT Filed: May 30, 2003
(86) PCT No.: PCT/AU03/00661

§ 371 Date: Apr. 3, 2006

Related U.S. Application Data
(60) Provisional application No. 60/384,882, filed on May 31, 2002.
(60) Provisional application No. 60/387,284, filed on Jun. 7, 2002.

(30) Foreign Application Priority Data
Jun. 19, 2002 (AU) PS 3078
Sep. 5, 2002 (AU) 2002951223
Sep. 5, 2002 (AU) 2002951222

(51) Int. Cl. C12Q 1/00; G06F 19/00; C12M 1/34

(52) U.S. Cl. 435/4; 435/287.1; 702/19

(57) ABSTRACT

The present invention relates to apparatus for recovering selected particles having predetermined properties from a number of particles. The apparatus includes a stimulation system, a detection system, a retriever and a control system. In use, the control system is adapted to activate the stimulation system to attempt to stimulate one or more of the particles and then detect at least one stimulated particle using detection system. Once a stimulated particle has been detected this is recovered using the retriever.

### BACKGROUND OF THE INVENTION

The present invention relates to a method and apparatus for recovering selected cells.

### DESCRIPTION OF THE PRIOR ART

The reference to any prior art in this specification is not, and should not be taken as, an acknowledgement or any form of suggestion that the prior art forms part of the common general knowledge in Australia.

It is often desirable to obtain cells having predetermined properties from a group of cells or tissue having a range of properties. One way in which this may be achieved is through the use of flow cytometry. Flow cytometry is used to quantitatively measure physical or chemical characteristics of particles in fluid samples, as they are presented, in single file, into a focused light beam.

In this case, the cells are labelled with markers, such as fluorescent markers, such that the markers couple only to cells having the desired predetermined properties. The labelled cells are then injected into a stream of fluid flowing through the cytometer. A light beam is focused onto the stream of fluid, such that as the cells pass through the light beam, the markers fluoresce, allowing the cells having the desired properties to be detected. The cells may then be sorted using either droplet deflection or a mechanical sorter.

In the case of droplet deflection, a piezoelectric transducer is used to create droplets of sheath fluid. An electric field is applied to the drops to sort them in accordance with the cells contained therein. Alternatively, a mechanical capture tube or the like may be inserted into the fluid flow to recover cells contained therein, as described for example, in U.S. Pat. No. 5,030,002.

Typically however flow cytometers are sophisticated instruments that require at least daily alignment by a highly skilled operator. Setting up the apparatus is also difficult and requires complex calibration.

Furthermore these techniques can generally only be used to sort large numbers of cell populations, however it is somewhat limited when the number of the cells within the population are low, or when only small cultures are available. FACS machines can operate (sort) down to around 1000 cells. The present invention intends to have the ability to operate down to single cells

However, as there are often only small numbers of cells having desired properties, it is necessary to have apparatus to detect, recover and isolate single cells in a fashion that is non detrimental to the cell. These cells are then available for further analysis.

### SUMMARY OF THE PRESENT INVENTION

In a first broad form the present invention provides apparatus for recovering selected cells from a number of cells, the selected cells having predetermined properties, the apparatus including:
a) A stimulation system;
b) A detection system;
c) A retriever; and,
d) A control system, coupled to the stimulation and detection systems and a pipette, the control system being adapted to:
   i) Attempt to stimulate one or more of the cells using the stimulation system;
   ii) Detect at least one stimulated cell using detection system, the stimulated cell having the predetermined properties; and,
   iii) Recover a stimulated cell using the pipette.

The retriever is typically a pipette, although other devices for retrieving the cells could be used.

The pipette is preferably coupled to a drive system, and includes an actuator adapted to actuate the pipette to thereby expel or draw in cells through a port. In this case, the control system is typically adapted to:
a) Operate the drive system to position port adjacent the stimulated cell; and,
b) Recover the cell by operating the actuator to thereby draw the cell into the pipette.

In this case, the drive system is typically a micromanipulator.

The detection system may be adapted to:
a) Determine the position of the stimulated cell; and,
b) Transfer an indication of the position of the stimulated cell to the control system, the control system being adapted to operate the drive system in accordance with the indicated stimulated cell position.

The stimulation system may be coupled to the pipette, thereby allowing the stimulation system to stimulate cells near the port.

The control system is typically operated to:
a) Operate the drive system until a number of cells are detected by the detection system;
b) Attempt to stimulate the cells; and,
c) Repeat steps (a) and (b) until one or more stimulated cells are detected.

The cells having the predetermined properties may be coupled to respective markers, in which case the stimulation system is typically adapted to stimulate the cells by stimulating the markers. However, direct stimulation of the cells may be possible in some circumstances.

The markers may be fluorescent markers, in which case the stimulation system typically includes a radiation source for stimulating the fluorescent markers. However, other markers, such as magnetic markers, or the like, may be used.

The radiation source is typically a laser, although LED or other radiation sources may be used.

Alternatively, the markers may be magnetic markers, in which case, the stimulation system is formed from a magnetic field generator adapted to generate a magnetic field.

The magnetic field can be adapted to attract stimulated particles towards the retriever for recovery.

The stimulated particles can be drawn into the pipette under the action of the magnetic field. Additionally or alternatively, standard use of the pipette can be used.

The detection system may be formed from the magnetic field generator, with the stimulated particles being determined by the attraction of the particles to the magnetic field.

The detection system typically includes:
a) An imaging system for generating images of one or more of the cells; and,
b) A processing system for detecting cells and/or stimulated cells in the images.

The imaging system is preferably being coupled to the drive system to thereby generate images of a region near the port. However, a separate respective drive system may be used.

The particles may be any form of particles, although the apparatus is particularly suitable for use with cells.

In a second broad form, the present invention provides a method of recovering selected cells from a number of cells, the selected cells having predetermined properties, the method including:
a) Attempt to stimulate one or more of the cells;
b) Detect at least one stimulated cell having the predetermined properties; and,
c) Recover a stimulated cell using a retriever.

The retriever is preferably a pipette, although other retrievers could also be used.

The pipette is typically being coupled to a drive system, and includes an actuator adapted to actuate the pipette to thereby expel or draw in cells through a port The method preferably includes:
a) Operate the drive system to position port adjacent the stimulated cell; and,
b) Recover the cell by operating the actuator to thereby draw the cell into the pipette.

Generally the method includes using a detection system to:
a) Determine the position of the stimulated cell; and,
b) Operate the drive system in accordance with the indicated stimulated cell position.

The method typically includes:
a) Using an imaging system for generating images of one or more of the cells; and,
b) Using a processing system for detecting stimulated cells in the images.

The method generally includes causing the control system to:
a) Operate the drive system until a number of cells are detected by the imaging system;
b) Attempt to stimulate the cells; and,
c) Repeat steps (a) and (b) until one or more stimulated cells are detected.

The markers may alternatively be magnetic markers, in which case the the method including applying a magnetic field to the particles to thereby stimulate the particles.

The magnetic field can be adapted to attract stimulated particles towards the retriever for recovery. In this case, the stimulated particles being drawn into the pipette under the action of the magnetic field.

The method can include detecting stimulation of the particles by detecting the attraction of the particles towards the retriever.

The method is preferably performed by apparatus according to the first broad form of the invention.

In a third broad form the present invention provides a processing system for controlling apparatus for recovering selected cells from a number of cells, the selected cells having predetermined properties, the apparatus including a stimulation system, a detection system and a retriever, the processing system being adapted to:
a) Attempt to stimulate one or more of the cells using the stimulation system;
b) Detect at least one stimulated cell using detection system, the stimulated cell having the predetermined properties, and,
c) Recover a stimulated cell using the retriever.

The processing system is typically being adapted to perform the method of the second broad form of the invention, and operate as the control system of the first broad form of the invention.

In a fourth broad form the present invention provides a computer program product for recovering selected cells from a number of cells, the selected cells having predetermined properties, the apparatus including a stimulation system, a detection system and a retriever, the computer program product including computer executable code which when executed by a suitable processing system causes the processing system to perform the method of the second broad form of the invention.

In a fifth broad form the present invention provides a pipette system for manipulating particles, the pipette system including:
a) A pipette for containing fluid in use, the pipette including a port;
b) An actuator coupled to the pipette, the actuator being adapted to draw in and/or expel fluid through the port;
c) A radiation source; and,
d) A waveguide having a first end coupled to the radiation source and a second end coupled to the pipette adjacent the port to thereby allow radiation from the radiation source to impinge on particles positioned adjacent to the port in use.

The pipette system typically includes a detector adapted to detect radiation emitted by the particle. In this case, the detector is preferably coupled to the first end of the waveguide, to thereby detect radiation emitted from the particle.

The radiation source may be a laser.

The waveguide can be a fibre optic cable, or alternatively may be formed from the pipette, the pipette including a shaped portion to allow the radiation from the radiation source to enter the pipette and pass along at least a portion of the pipette, the radiation being emitted from the pipette through the port.

The pipette system typically includes a controller adapted to perform at least one of:
a) Activating the actuator to thereby cause fluid to be drawn in and/or expelled through the port; and,
b) Activating the radiation source, to thereby expose a particle to radiation.

The pipette system typically includes a drive system adapted to move the pipette system to be with respect to a fluid filled container to thereby allow particles to be positioned in or removed from fluid in the container.

The drive system may be coupled to a controller, the controller being adapted to recover particles having predetermined properties from the container by:
a) Positioning the pipette system such that the port is adjacent to a particle;
b) Activating the radiation source to thereby expose the particle to radiation;
c) Detect any radiation emitted by the particle;
d) Determine if the particle has the predetermined properties in accordance with the detected radiation; and,
e) In accordance with a successful comparison, activate the actuator to thereby draw fluid into the pipette through the port, thereby recovering the particle.

The controller is preferably formed from a suitably programmed processing system.

In a sixth broad form the present invention provides a pipette system for manipulating particles, the pipette system including:
a) A pipette for containing fluid in use, the pipette including a port;
b) An actuator coupled to the pipette, the actuator being adapted to draw in and expel fluid through the port, the actuator including:
   i) A fluid reservoir;
   ii) A flexible tube coupling the pipette to the fluid reservoir;
   iii) An arm positioned so as to partially compress the tube;
   iv) An actuator drive system adapted to move the arm so as to perform at least one of:
      (1) Further compressing the tube to thereby expel fluid from the port; and,
      (2) Decompressing the tube to thereby draw fluid in through the port.

The actuator drive system typically includes:
a) A first actuator drive for moving the arm with respect to the tube; and,
b) A second actuator drive formed from an arm end portion, the arm end portion being in contact with the tube in use, the second actuator drive being adapted to cause the arm end portion to expand or contract.

The pipette system typically includes a controller coupled to the actuator drive system, the controller being adapted to operate the actuator drive system to thereby draw fluid in or expel fluid through the port.

The pipette system preferably includes a drive system adapted to move the pipette system to be with respect to a fluid filled container to thereby allow particles to be positioned in or removed from fluid in the container.

The drive system is typically coupled to the controller, the controller being adapted to recover particles from the fluid by:
a) Positioning the pipette system such that the port is adjacent to a particle; and,
b) Activate the actuator drive system to thereby draw fluid into the pipette though the port, thereby recovering the particle.

The tube may be formed from silicon tubing.

Typically the pipette system according to the sixth broad form of the invention is a pipette according to the fifth broad form of the invention.

In a sixth broad form the present invention provides a pipette system for manipulating particles, the pipette system including:
a) A pipette for containing fluid in use, the pipette including a port;
b) An actuator coupled to the pipette, the actuator being adapted to draw in and/or expel fluid through the port; and,
c) A magnetic field generator for exposing particles to a magnetic field.

The magnetic field generator may be formed from a solenoid, or the like.

The pipette can include a nozzle, in which case the solenoid is formed from a graphite layer and a number of associated windings arranged on the nozzle.

The pipette system may include a controller adapted to perform at least one of:
a) Activating the actuator to thereby cause fluid to be drawn in and/or expelled through the port; and,
b) Activating a current supply coupled to the solenoid, to thereby expose the particles to a magnetic field.

The pipette system typically includes a drive system adapted to move the pipette system to be with respect to a fluid filled container to thereby allow particles to be positioned in or removed from fluid in the container.

### BRIEF DESCRIPTION OF THE DRAWINGS

An example of the present invention will now be described with reference to the accompanying drawings, in which:

FIG. 1 is a block diagram of an example of apparatus for implementing the present invention;

FIG. 2A is a schematic diagram of the pipette of FIG. 1;

FIG. 2B is a schematic diagram of the operation of the actuator of FIG. 2A;

FIG. 2C is a schematic diagram of a first example of the pipette of FIG. 2A modified for use with a bladder;

FIG. 2D is a schematic diagram of a second example of the pipette of FIG. 2A modified for use with a bladder;

FIG. 3 is a schematic diagram of the stimulation system of FIG. 1;

FIG. 4 is a schematic diagram of the apparatus of FIG. 1;

FIGS. 5A to 5C are a flow chart of the process implemented by the apparatus of FIG. 1;

FIGS. 6A to 6E are schematic diagrams of cells in the selection and recovery wells of FIG. 4;

FIGS. 7A and 7B are schematic diagrams of cells being drawn into and expelled from the pipette of FIG. 2;

FIG. 8 is a schematic diagram of the distribution of cells into a well plate;

FIG. 9 is a schematic diagram of the pipette nozzle holding a number of cells;

FIG. 10 is a schematic diagram of an example of an alternative pipette actuator; and,

FIG. 11 is an example of the pipette of FIG. 2 modified to include an electrode;

FIG. 12 is an example of a modified pipette; and

FIG. 13 is an example of a suitable cutting tool.

### DETAILED DESCRIPTION OF THE PREFERRED EMBODIMENTS

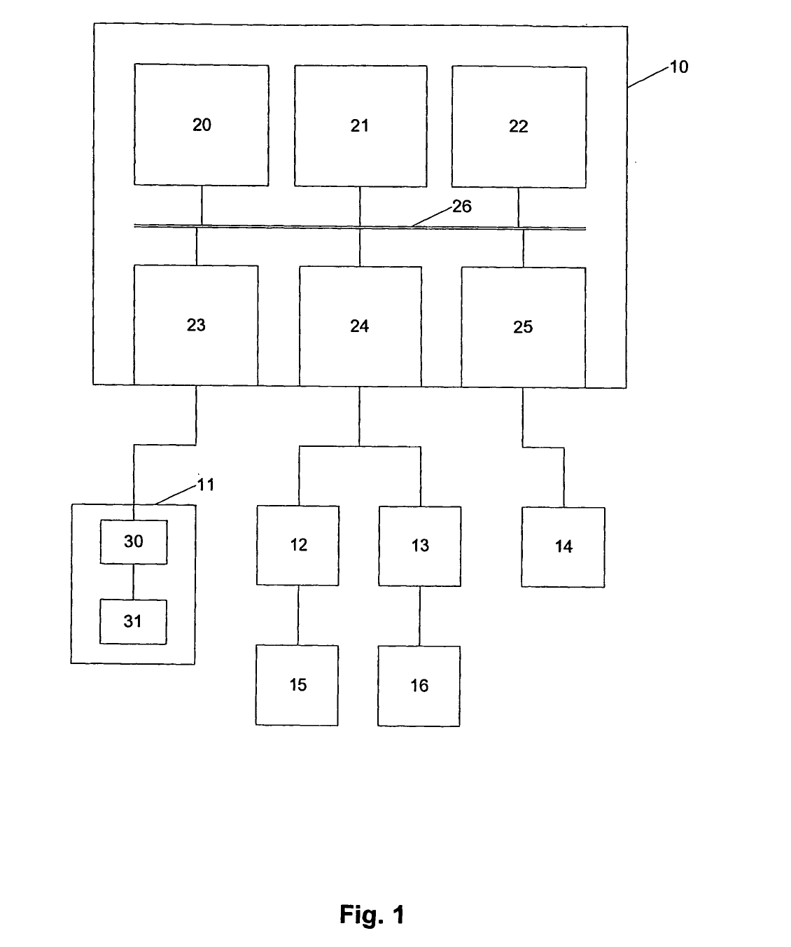

An example for apparatus suitable for implementing the present invention will now be described with reference to FIGS. 1.

As shown in FIG. 1, the apparatus includes a processing system 10 coupled to an imaging system 11, a first drive system 12, a second drive system 13 and a stimulation system 14. The first drive system 12 is coupled to a pipette 15, with the second drive system being coupled to a stage 16, as shown.

The processing system 10 includes a processor 20, a memory 21, an input/output (I/O) device 22, an image interface 23, a drive interface 24, and a stimulation interface 25, coupled together via a bus 26. The processing system may therefore be any one of a number of processing systems, such as a suitably programmed computer, specialised hardware, or the like.

In any event, the I/O device typically includes a display, such as a computer monitor or the like, a keyboard, and one or more other input devices such as a mouse, joystick, trackball or the like.

The imaging system 11 includes a camera 30 such a CCD camera or the like which is coupled to a microscope 31. The imaging system 11 is connected to the processing system via the image interface 23.

In use, the drive systems 12, 14 are coupled to the processor via the drive interface 24, thereby allowing the processor 20 to control motion and operation of the pipette 15 and the stage 16, as will be described in more detail below. Similarly, the stimulation system 14 is coupled to the stimulation interface 25, to allow the stimulation system to be activated, as will be described in more detail below.

In use, this allows cells having predetermined properties to be recovered from a group of cells held in suspension in a suitable container. In order to achieve selection the cells are labelled with markers, which are adapted to adhere and or permeate only the cells having the required predetermined properties. The processing system 10 can then activate the stimulation system 14 to stimulate the marker cells and thereby identify the cells having the predetermined properties.

It will be appreciated that this may be achieved in a number of ways depending on the characteristics of the markers, and the stimulation system. Thus for example, the markers could be magnetic markers, with the stimulation system being adapted to generate a magnetic field. This could be adapted to cause movement of the markers, and hence the cells having the predetermined properties, thereby allowing the cells to be identified. Alternatively, optical markers may be used, as will be described in more detail below.

In any event, once the cells having the predetermined properties have been identified, the processing system 10 can then control the pipette 14 to remove cells from the well 40. This may be achieved automatically or manually in accordance with input commands received from the user via the I/O device 22.

In order to achieve this, the processor 20 executes appropriate application software, which is stored in the memory 21, to control the operation of the apparatus.

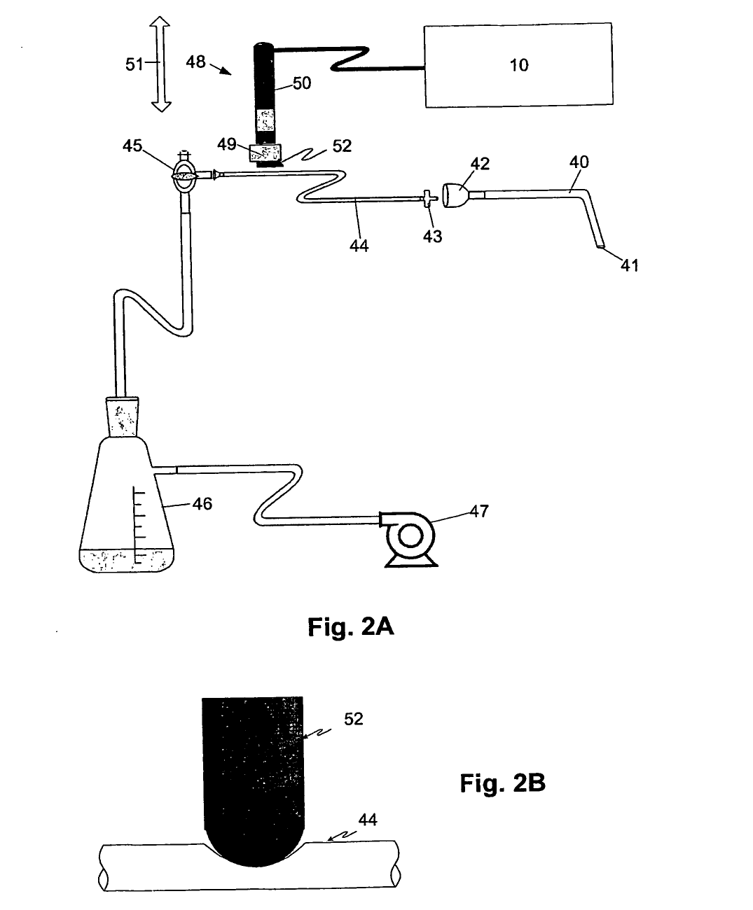

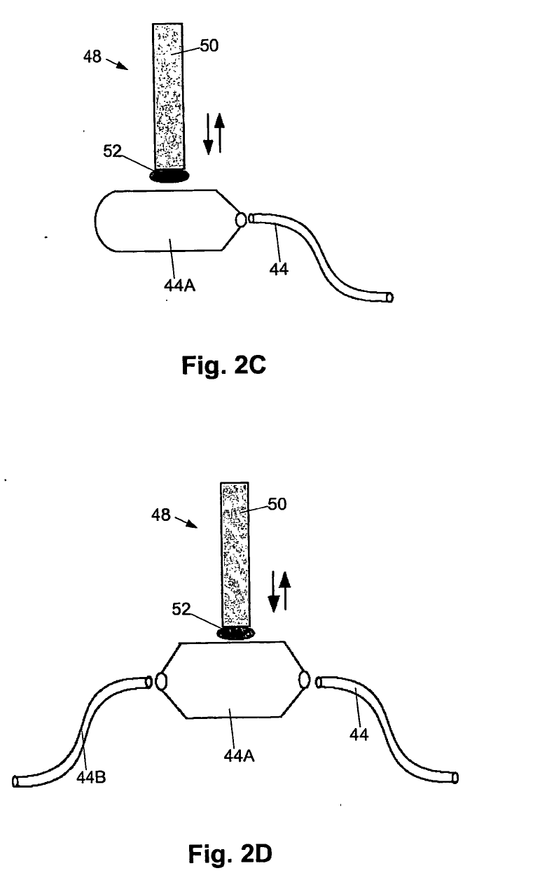

A detailed example will now be described with reference to FIGS. 2 to 7.

The pipette 14 is shown in more detail in FIG. 2A. As shown, the pipette is formed from a glass nozzle 40 having a port 41. The glass nozzle includes a female coupling 42 that is adapted to cooperate with a male coupling 43 on a flexible tube 44. In use, the tube 44 is connected via a stopcock 45 and a reservoir 46 to a pump 47. An actuator 48 is positioned adjacent the flexible tube 44, to allow the tube to be clamped as shown in FIG. 2B.

It will be appreciated from this that any form of actuator, such as a solenoid, may be used. However, in this example, the actuator is formed from a threaded screw drive 49, coupled a DC or stepper motor 50, which forms part of the drive system 12. In use, this allows the actuator to be moved in the direction of the arrow 51, an amount of ±5 mm.

The actuator tip can have a piezo electric stack 52 coupled thereto, to allow fine control (displacement of ±20 μm) of the end of the actuator. Again, the piezo stack forms part of the drive system 12.

In use, the pipette is loaded with a suitable fluid medium by placing the port 41 into a container that has sufficient fluid to fill the system. The pump or other such means of drawing fluid through the system 47 is activated and fluid is drawn through the pipette. When the system is loaded and there are no air bubbles present in the tubing, the stopcock 45 is closed to prevent further fluid flow, and the pump 47 turned off.

Whilst the port 41 is still immersed in the fluid medium, the actuator 48 is adjusted such that the silicon tubing is compressed to about half its diameter, as shown in FIG. 2B. Thus, in use, with the port 41 positioned in fluid in a well causing the actuator 48 to move in the direction of the arrow 51 compresses or releases the tubing 44 which, in turn, either expels or draws in fluid through the port 41. This allows cells to be recovered from a well, as will be explained in more detail below.

Variations on this design are shown in FIGS. 2C and 2D. In these examples, the actuator 48 is positioned adjacent a bladder 44A provided in the flexible tube 44. In this case, the bladder has a larger cross sectional area than the tube and will therefore contain a greater volume of fluid per unit length compared to the tubing 44. This has two main benefits. In particular, the larger cross sectional area provides for a greater range of movement of the actuator. This coupled with the increased fluid volume in the bladder allows for a greater amount of fluid to be displaced when compared to the action of the actuator on the tube 44.

As a result this provides greater control over the amount of fluid expelled or drawn in through the aperture 41, allowing for greater accuracy in retrieving individual cells using the pipette.

In this instance, it will be appreciated that by providing sufficient liquid in the bladder, it is not necessary to provide the stopcock 45, the reservoir 46 or the pump 47, as shown in FIG. 2C. In particular, the bladder and pipette can be filled, with an amount of fluid being expelled from the bladder before the bladder is positioned so as to cooperate with the actuator, thereby allowing the actuator position to be adjusted to allow fluid to be drawn in or expelled through the aperture 41.

Alternatively, the bladder can be connected to a stopcock 45, reservoir 46 and pump 47, by a tube 44B, as shown in FIG. 2D.

For the remainder of the description, the example will focus on the use of the actuator on the tube 44, although it will be appreciated that the techniques will also apply to the actuator acting on an appropriate bladder.

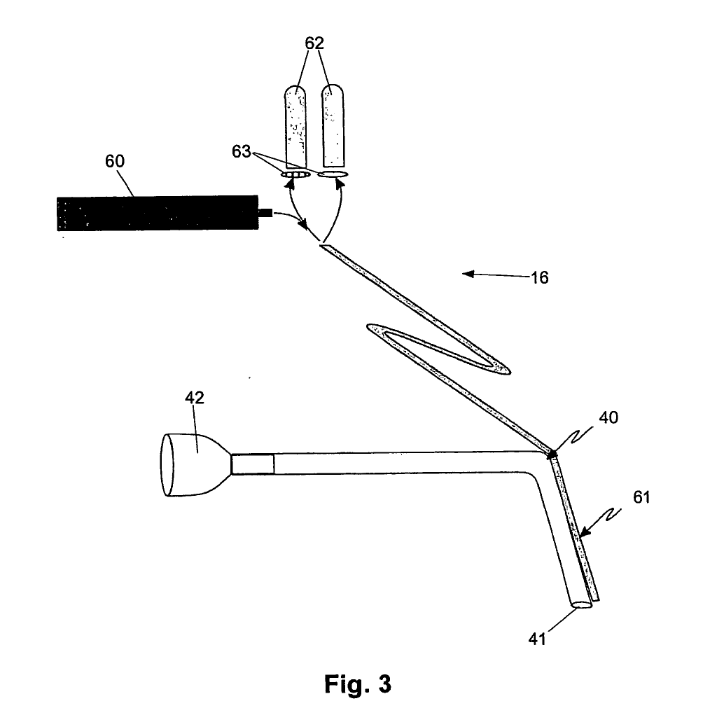

In this example, the stimulation system 14 is coupled to the pipette 15, as shown in more detail in FIG. 3. As shown, the stimulation system includes a radiation source 60, such as a UV burner with suitable filters, a laser, or the like, coupled to an optical fibre 61.

The optical fibre is coupled to the pipette nozzle 40, using appropriate fixing means, such as a rubber tube (not shown). The optical fibre is also coupled to detectors 62, such as photo-diode tubes, via suitable filters 63, to detect emissions from cells, as will be explained in more detail below.

In use, the system can select and remove individual cells using the pipette 15 from a group of cells held in suspension.

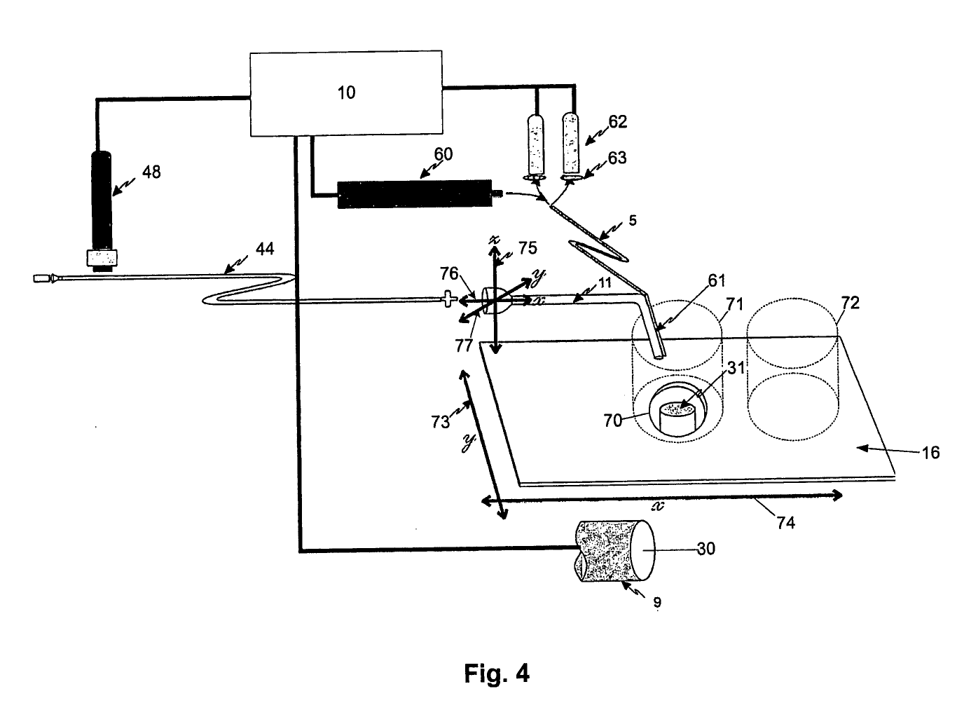

In order to achieve this, the apparatus is arranged as shown schematically in FIG. 4. As shown, the stage 16 includes an aperture 70, having the microscope 31 mounted therein. From this it will be appreciated that the microscope 31 is typically an inverted microscope.

In use the stage 16 is adapted to receive a selection well 71 containing the cells to be recovered. The stage may also receive a recovery well 72 for receiving the recovered cells. In use, the selection well 71 is positioned on top of the aperture 70, to allow the camera 30 to obtain an image of the inside of the selection well 71, via the microscope 31. In use, the processing system 10 is adapted to control the drive system 14, to cause the stage to be move in the directions shown by the arrows 73, 74.

This allows a representation of the contents of a selected well can be captured by the processing system using the image interface, which is typically a flame grabber or the like. Images may then be used by the processing system to control the drive system 12 and the stimulation system 13. Additionally or alternatively, images may be displayed to a user using the I/O device 22.

The pipette is positioned adjacent the stage 16 as shown, to allow the nozzle to be inserted into the well 71. The pipette is coupled to the drive system 12, to allow the pipette to moved with respect to the well, as shown by the arrows 75, 76, 77. Accordingly, the drive system 12 typically includes a micromanipulator system having three independently controlled axis with resolution tolerances and repeatabilities of <5 μm. This system is controlled by dedicated software executed by the processor 20.

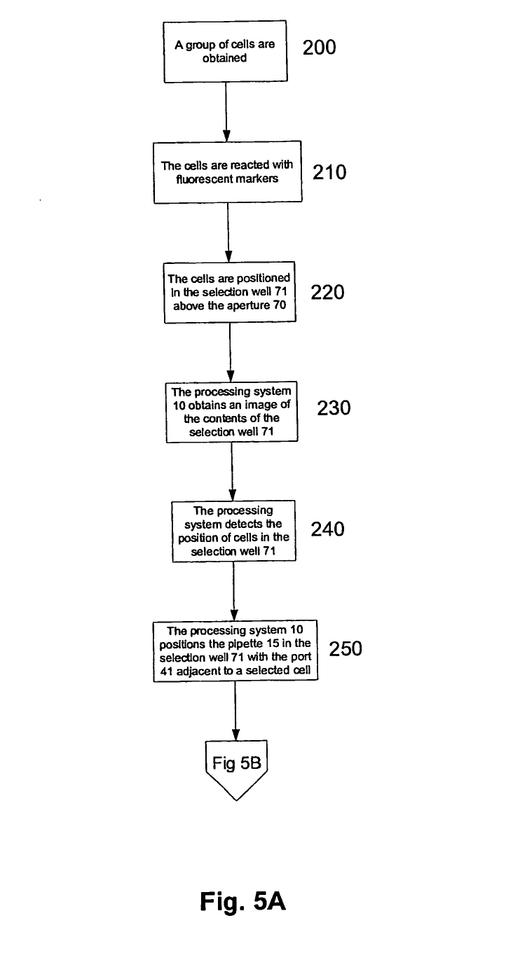

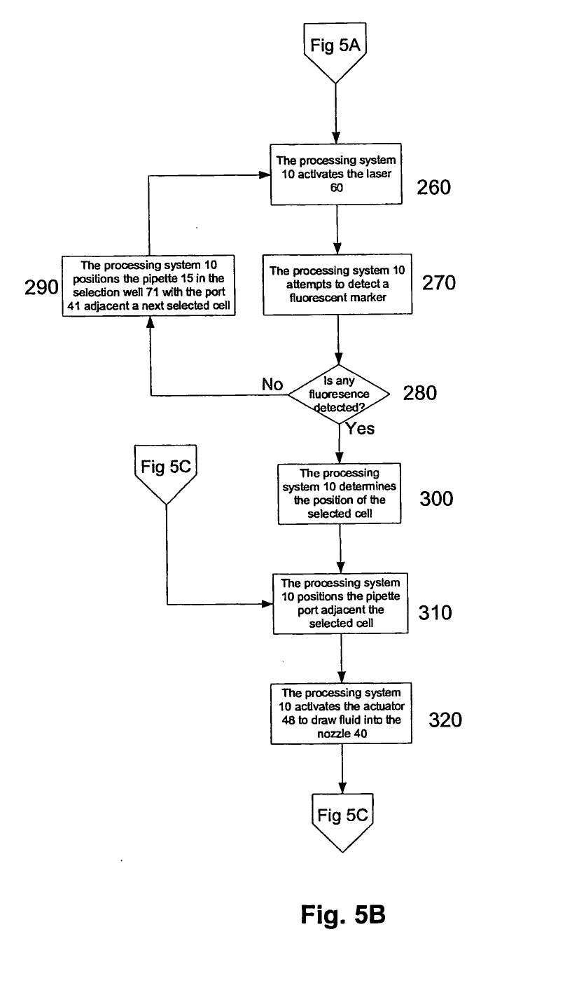

An example of the method of operation of the apparatus of FIG. 4 to recover selected cells will now be described in detail with reference to FIGS. 5A, 5B and 5C.

In particular, the process involves obtaining a group of cells at step 200, with at least some of the cells having predetermined properties that are of interest to the user of the apparatus.

At step 210 the user labels the cells with fluorescent markers, such that each cell having the predetermined properties becomes labelled with a respective marker, whilst cells having different properties do not As a result of this, only cells having the predetermined properties are labelled the fluorescent markers.

At step 220 the labelled cells are placed in the selection well 71 above the aperture 70, as shown in FIG. 4. It will be appreciated therefore that the cells may therefore be labelled with the markers whilst the cells are held in suspension in the selection cell 71, allowing the selection well to be placed above the aperture as required.

In any event, the processing system 10 then uses the camera 30 to obtain an image of the cells within the selection well 71, at step 230, using the image to determine the position of the cells at step 240. This is achieved by having the imaging system 30 generate a sequence of images of the inside of the selection well 71. The image interface then selects an appropriate one of the images and transfers this to the processor 20. The processor 20 will then analyse the image to determine the position of the cells. In particular, the processor 20 will typically use edge detection software to detect edges in the image representing the edge of the cells in the solution.

At step 250 the processing system 10 activates the drive system 12 causing the pipette 15 to be positioned in the selection well 71, with the port 41 adjacent to and above a selected cell. In particular, the pipette 15 is positioned such that radiation emitted from the fibre optic cable 61 impinges on the selected cell.

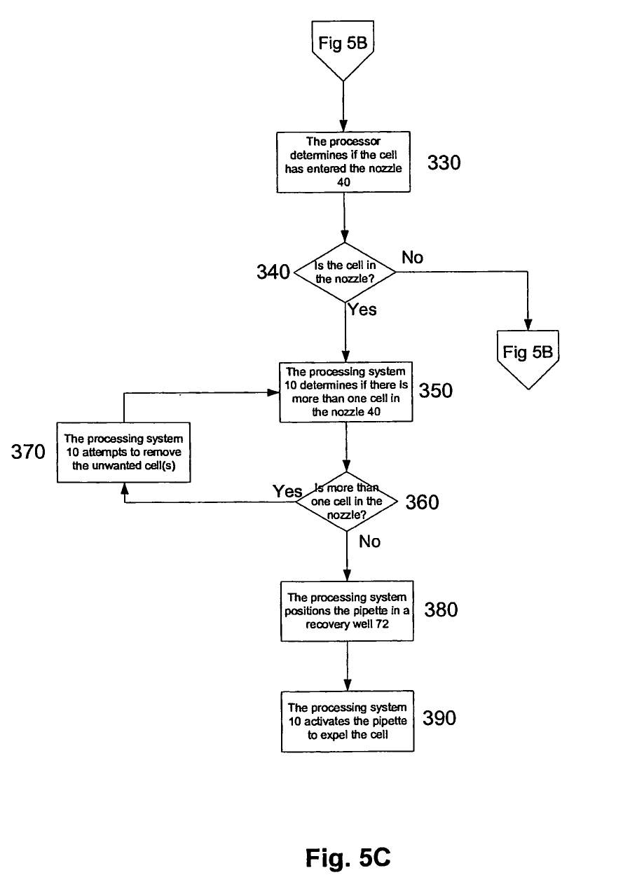

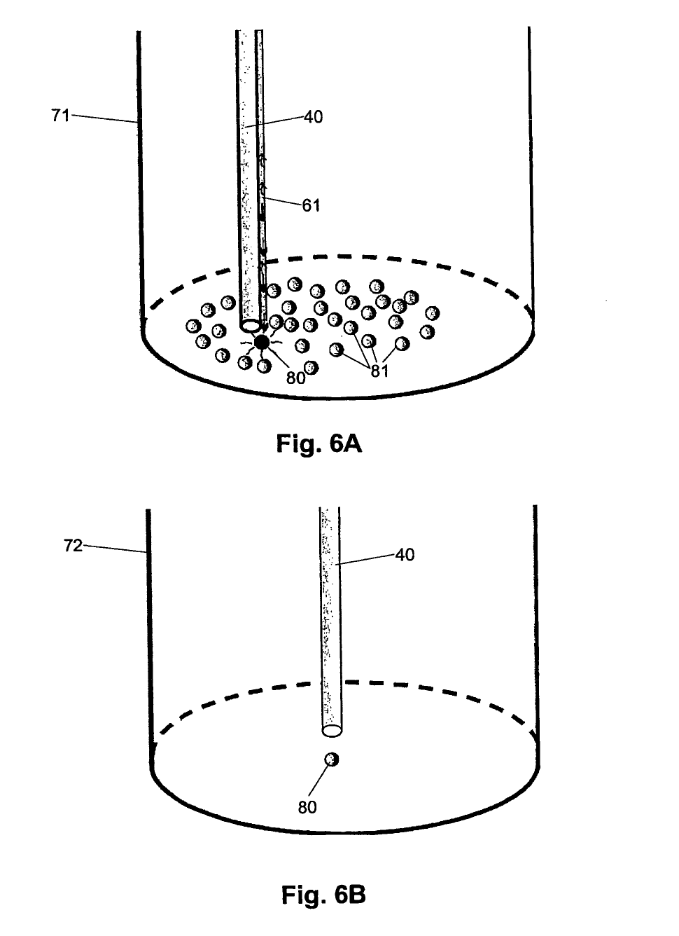

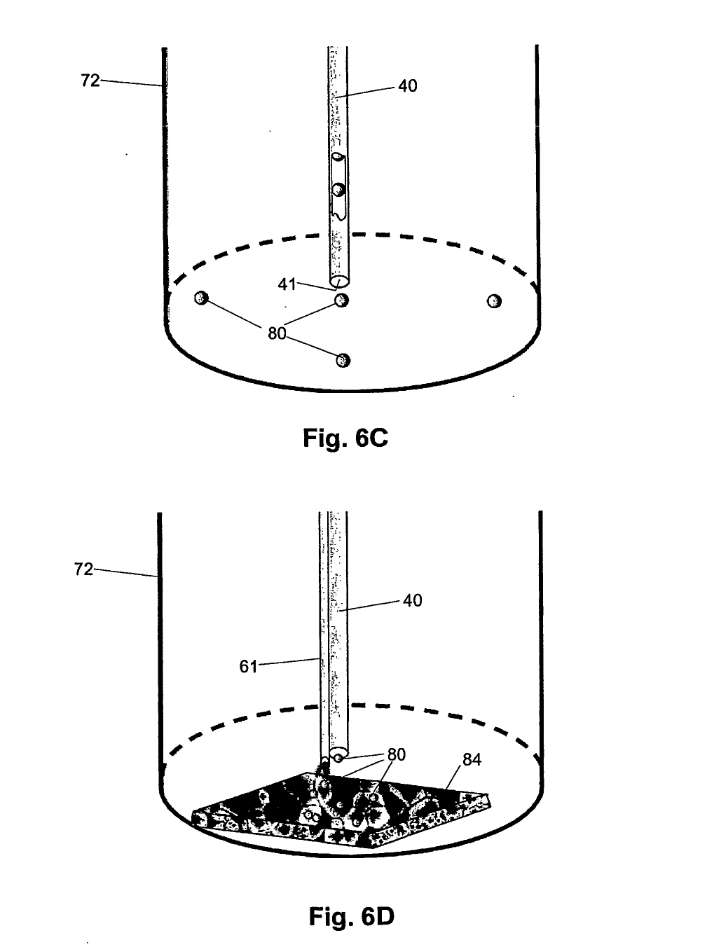

At step 260, the processing system 10 activates the laser 60, causing radiation to be emitted from the fibre optic cable 61. In particular, the laser is activated such that the radiation from the laser impinges on the selected cell. It will be appreciated that if the cell has the predetermined properties, the marker bound to the cell will fluoresce under the influence of the radiation. An example of this is shown, for example in FIG. 6A, in which a selected cell 80, amongst other cells 81, is fluorescing.

At step 270, the processing system uses the detectors to detect any fluorescent markers, by examining for fluorescence in the image. If no fluorescence is detected at step 280, then this indicates that no marker has been exposed, and hence that the cell does not have the predetermined properties.

Accordingly, the processing system moves the position of the pipette 15 and hence the position of the fibre optic cable 61, such that different cells are positioned adjacent the end of the fibre optic cable. The processing system then repeats step 260 to 280, so that different cells are exposed to radiation.

This process is repeated until a fluorescent marker is detected, allowing the processing system 10 to select a cell that is suitable for recovery.

At step 300 the processing system 10 determines the position of the selected cell. At step 310 the processing system 10 positions the port 41 adjacent the selected cell. This is achieved by using feedback to monitor the position of the port 41 as the drive system 12 is activated, as will be appreciated by those skilled in the art

At step 320 the processing system 10 activates the actuator 48, as described above, to draw fluid into the port 41. At this stage, the drawing in of the fluid should cause the cell to be drawn into the pipette nozzle 40. At step 330, the processing system operates to determine if the cell has entered the nozzle 40.

This could be achieved for example by having the camera 30 image the inside of the pipette nozzle 40. Alternatively, the processing system 10 may be adapted to track the cells in the selection well 71, by comparing subsequent images captured by the camera 30. This allows the processing system to track movement of the cells and determine when a respective cell has been removed from the selection well 71 and hence is contained within the pipette nozzle 40.

If it is determined that the cell is not in the nozzle at step 340, the processing system 10 returns to step 310 to reposition the pipette port adjacent the selected cell. The processing system 10 then repeats steps 310 to 340 until it is determined that the cell is in the nozzle 40.

At step 350 the processing system determines if any other cells are also contained within the nozzle 40. This may occur for example if two cells are positioned adjacent each other in the selection well 71. In particular, when the fluid is drawn into the pipette nozzle 40, this can cause multiple cells to be drawn in through the port.

It will be appreciated that this may be desirable if all the cells in the nozzle have the predetermined properties, in which case the processing system can simply move onto step 380. However, if cells that don't have the predetermined properties are recovered, this could contaminate the group of cells that are eventually recovered.

Accordingly if the processor determines that more than one cell is included in the pipette at step 360 the processing system 10 will attempt to remove one of the cells at step 370. This can be achieved by repeatedly operating the pipette to cause the pipette to repeatedly draw in and expel fluid via the pipette aperture 41. Agitation of the fluid medium and repeated movement of the cells through the pipette aperture 41 will usually allow a cell to be separated from surrounding cells.

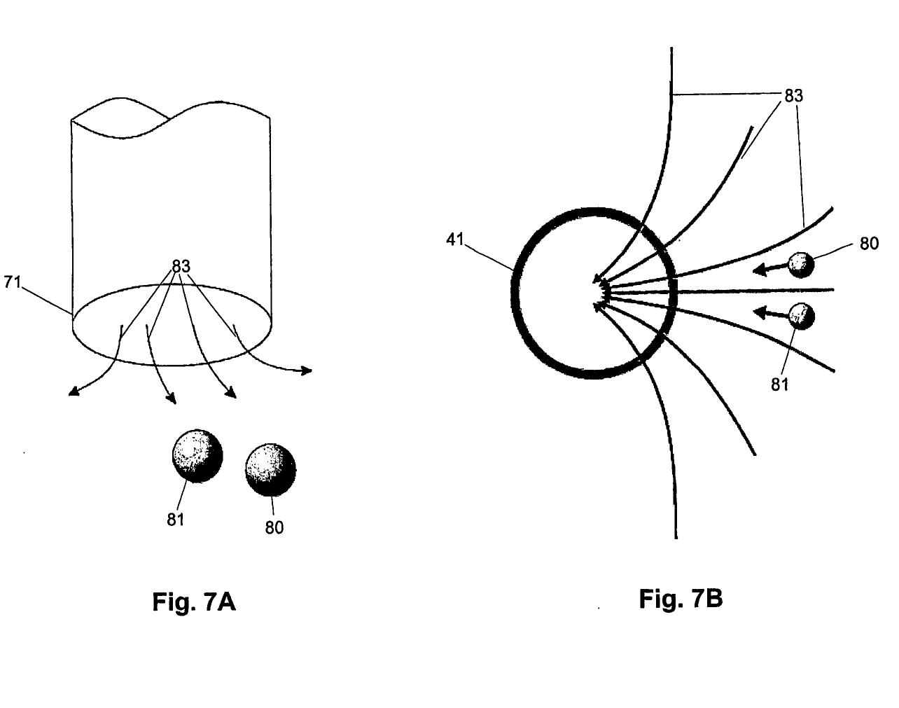

An example of this is shown in FIG. 7A, which shows the hydrodynamic stream-lines 83 as fluid is expelled from the pipette aperture 41. As shown, the hydrodynamic stream-lines, which represent lines of constant force, spread out away from the pipette aperture 41. Similarly, as the selected cell and unwanted cell 80, 81, are entrained in the fluid flow, this will tend to cause the cells 80, 81 to separate as they are expelled away from the pipette aperture 41.

Accordingly, repeated activation of the pipette allows the cells 80, 81, to be separated, allowing the selected marked cell to be collected alone.

At step 380 the processing system 10 re-positions the pipette in a recovery well 72 and activates the pipette to expel the cell into the recovery well 72 at step 390.

It will be appreciated from this that this provides a system for automatically recovering cells having predetermined properties. In particular, the apparatus can be provided with cells in the selection well 71 and then left to operate to automatically remove cells 80 having the desired properties, to the recovery well 72, as shown in FIG. 6B.

By repeating this procedure, this allows a large number of cells 80 having predetermined properties to be recovered to the recovery well 72. During this procedure, the processing system 10 can be adapted to distribute the recovered cells 80 in a predetermined pattern throughout the recovery well, as shown for example in FIG. 6C, or FIG. 6D.

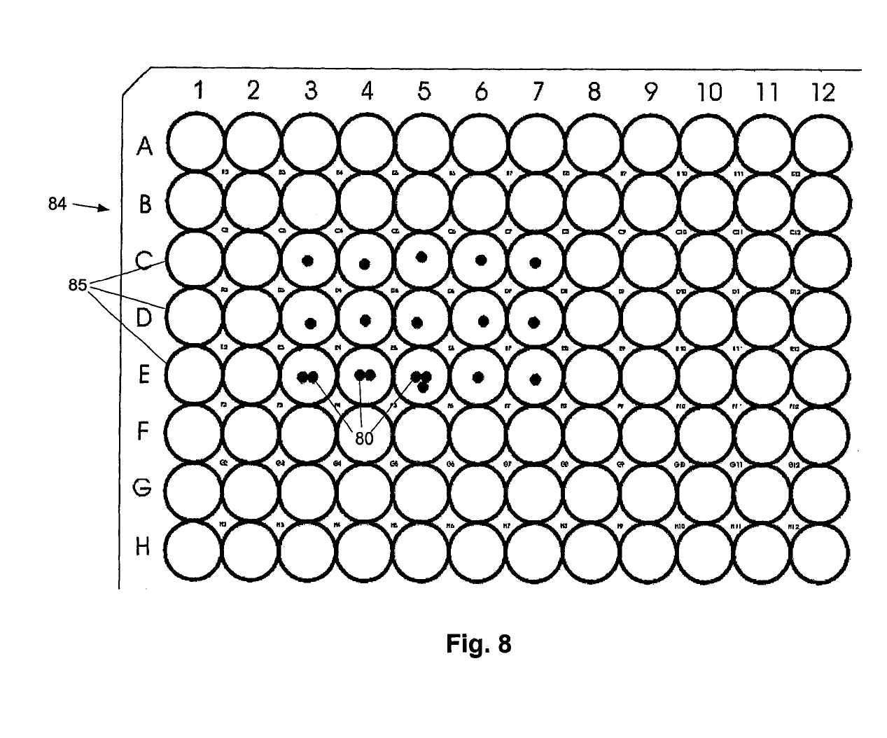

Alternatively, individual cells may be positioned in different recovery wells, as will be appreciated by persons skilled in the art. An example of this is shown in FIG. 8, in which the cells 80 are distributed into a well plate 84, including a number of recovery wells 85 arranged in a grid hike fashion. This allows the cells 80 to be positioned in the recovery wells 85, either individually, or with multiple cells per recovery well, as shown.

In general, the recovery well will include a growth medium to encourage growth of the recovered cell. However, the recovery well may instead include a localised section of the culture/tissue, as shown for example at 84 in FIG. 6E. Microinjecting the recovered cell directly into the culture, as shown in FIG. 6E, can further aid cell recovery.

A particular benefit of this process is that cells are recovered on an individual basis. This allows cells having very strict criteria to be collected. As cells are collected on an individual basis, this prevents the opportunity of contamination of a sample cell in the recovery well by cells not having the required properties. Furthermore, this allows a larger number of cells to be recovered automatically.

Variations

A number of variations on the above are possible.

Stimulation

It will be appreciated that it may not be necessary to use markers, if the cells having the predetermined properties can be distinguished from other cells in the group using some other techniques. Accordingly, if the cells have different properties, this would allow direct stimulation of the cells to distinguish those having the required predetermined properties.

These properties may include optical and magnetic properties. However, in addition to this, other properties, such as dimensional properties may also be used to distinguish the cells. In this instance, the stimulation will involve using laser measurements to allow the dimensions of the cells to be determined.

Pipette

It will be appreciated as well that the pipette can be used to provide additional functionality. Thus, for example, the pipette could be used to remove fluid from a well and replace it with fresh/different media.

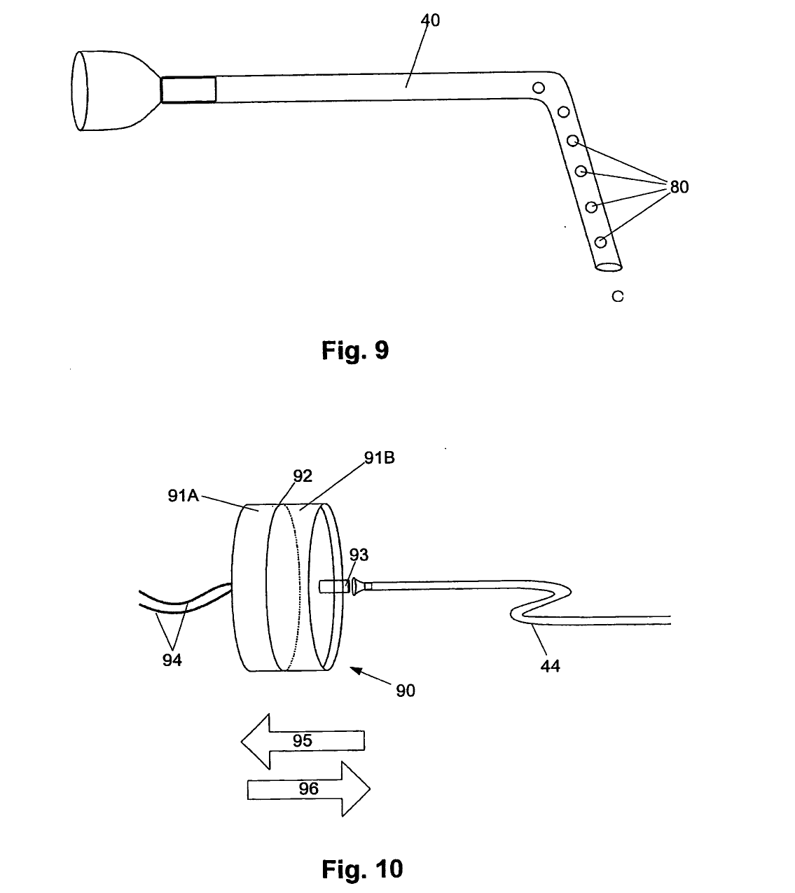

A further variation is for the processing system to collect a number of cells 80 having the predetermined properties from the selection well 71, with the cells being stored in the nozzle 40, as shown for example in FIG. 9. The cells can then be placed into one or more recovery wells 72, individually as required.

A number of other pipette modifications are also shown in our copending patent application entitled “Cell Fusion” and persons skilled in the art will appreciate that these modifications may be incorporated with each other.

Thus for example, the pipette system may use an alternate actuator to the actuator 48 shown in FIG. 2. This may include a piezo-electric actuator, shown for example in FIG. 10.

As shown, the actuator is formed from a housing 90 defining a chamber that is divided into two portions 91A, 91B by a piezoelectric element 92, as shown. The chamber 91B is coupled by a port 93 to the flexible tube 44, of the pipette shown in FIG. 2.

In use, the chamber 91B, the port 93, and the flexible tube 44, and the nozzle 40, are filled with fluid, with the chamber 91A being filled with air and sealed. Applying a current to the piezo-electric element 92, via leads 94, causes the element to move, with the direction of movement depending on the polarity of the applied current.

Thus, in use, with the pipette port 41 positioned in a fluid filled well, causing the piezoelectric element 92 to move in the direction of the arrow 95 will increase the volume of the chamber 91B, thereby causing fluid to be drawn through the port 41. Similarly, causing the piezoelectric element 92 to move in the direction of arrow 96 will decrease the volume of the chamber 91B, thereby causing fluid to be expelled through the port 41.

Accordingly, the pipette can be activated to draw in or expel fluid through the port 41 depending on the polarity of the current applied to the leads 94. Accordingly, in use, the leads 94 are coupled to the processing system 10, to allow a suitable signal to activate the pipette as required.

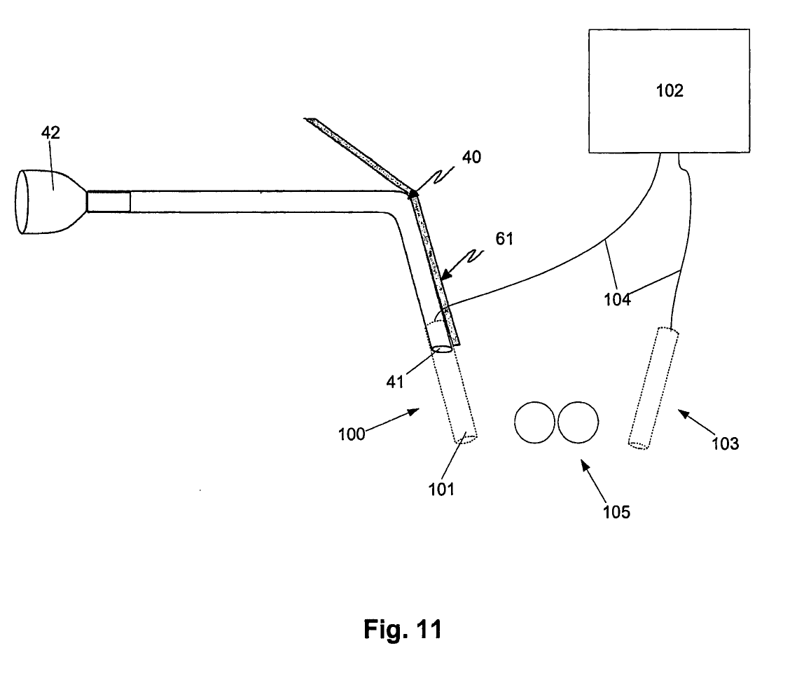

Similarly, the pipette may be adapted to incorporate an electrode, for use in apply an electric field to the cell, as used for example in cell dielectropherisis (DEP). And cell fusion techniques. An example of this shown in FIG. 11.

As shown, an electrode 100 formed from a cylindrical tube 101 is coupled to the nozzle 40 of the pipette 40, such that the port 41 is contained in the tube 101. In use, the pipette may be used substantially as described with respect to the pipette of FIG. 2.

Additionally however, the electrode 100 can be coupled to a field generator 102, which is also coupled to a second electrode 103, as shown by the leads 104. In use, the electrodes 100, 103 cooperate to allow electric fields to be applied to one or more cells 105, positioned therebetween. It will be appreciated that the electrode 103 may be formed from an electrode coupled to another pipette.

Magnetic Labelling

As mentioned above techniques exist for labeling cells that allows them to be magnetically sorted. In this example, meta beads are used as markers to identify cells of interest. This is achieved by that cells having desired properties can be fused to the beads and thereby extracted from a mixture of cells.

This can be achieved for example by coating he beads with an antibody of interest and then the beads into a culture of cells. Cells that are expressing the appropriate receptor on the surface bind to the beads. The culture is then filtered though a tube, placed in an external magnetic field containing thousands of small beads that attract and hold the labelled cells, whilst allowing the labelled cells to be washed through and discarded. Once the external magnetic field is removed the bound cells can then be washed through the tube and isolated as desired.

It will be appreciated that this may be achieved on a smaller scale using a pipette modified to incorporate an electromagnet.

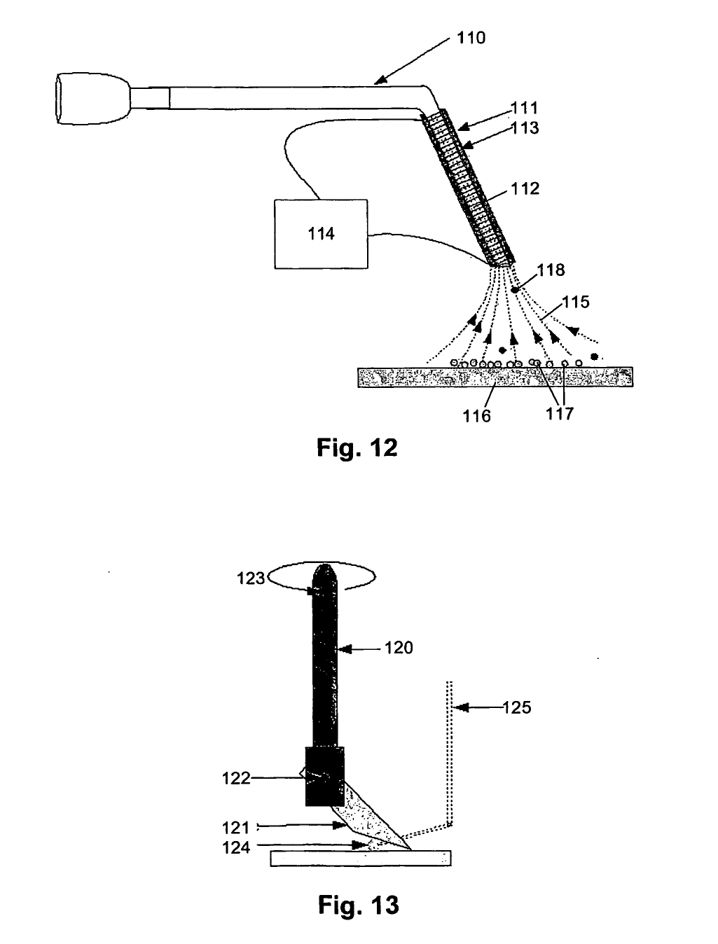

An example of a suitably modified pipette will now be described with reference to FIG. 12. In this example, the pipette shown generally at 110 includes a graphite layer 111 positioned around the pipette nozzle 112. A number of coper windings 113 are provided around a graphite core to form an electromagnet In use the copper windings are coupled to a DC power supply shown generally at 114, so that the Sings act as a solenoid to generate a magnetic field represented by the field lines 115.

The copper windings may be provided in a number of layers depending on the implementation, and may be embedded in a layer of epoxy in order to prevent electrolysis from occurring. The graphite layer 111 acts as a paramagnetic substance increasing the strength of the magnetic field within the pipette.

The ends of the wire arc connected to a variable DC power supply and a resistor (R). Passing a current trough the wire (taking account of Lenz's Law) will induce a magnetic field, the stray of which is proportional to the applied DC Voltage (V), as given by the equation:

$$B = nuI = \frac{nuV}{R}$$

where n=the number of turns per unit length
u=the permeability of free space.

In use, the pipette is positioned near a number of cells which may suspended in a fluid medium or resting on a substrate 116 as shown at 117. In this case, at least some of the cells are attached to appropriate magnetic m such as the beads outlined above.

In use, the metal poles, and hence the cells they are attached to, will be attracted into the magnetic field and can therefore be drawn into the pipette in the normal way. This allows cells coupled to the magnetic markers, ad hence cells having certain properties to be selected

It will be appreciated that cells with a higher density of receptors (a higher number of magneto markers), should have a lager force exerted on them than cells with less receptors for the same magnetic field strength. Therefore as the DC voltage is increased, a larger number of cells should be drawn into the magnetic fields influence. This field gradient can allow for a further sorting criteria.

In order to ensure no wanted cells have been collected, it is possible to flush out the pipette by expelling fluid from the nozzle. In this case, any cells not bound magnetic markers will be expelled from the pipette together with the fluid, whilst the cells bound to markers will be held in place by the action of the magnetic field. In this case, when the selected cells are to be expelled, the magnetic field can be deactivated, allowing the cells and attached markers to be expelled in the normal way.

Cutting

A further development that can be utilised within the examples described above is for a cutting tool to be provided to allow cells to be cut, as well as to allow cells that adhered to the well surface or electrodes to be released. An example of a suitable cutting tool is shown in FIG. 13. As shown, the cutting tool includes a support post 120 having a blade 121 pivotally mounted thereto by a binge 122 or other appropriate connection.

In use, the post is coupled to a micro manipulator (not shown), to allow the post to be positioned within the respective well. The post can be rotated as shown by the arrow 123, allowing the blade to be positioned above a cell to be cut. If the cell is a free cell 124, the cell will generally be held in place using a pipette, or other suitable manipulator, as shown at 125.

Once positioned, the post is lowered such that the tip of the blade ‘bites’ into the soft plastic of the bottom of the plastic plate. Further lowering of the post will cause the blade to pivot around the hinge 122 and ‘guillotine’ through object, such as the cell, placed in its path. Motion is stopped when the blade has cut through the object of interest and is completely parallel with bottom of plate.

It will be appreciated that the functionality of the different examples described above may be combined in any one of a number of arrangements. This allows for example cells to be selected automatically in accordance with magnetic or radiation sensitive markers. The cells can then be arranged in a fusion well, and fused, with the fusate being automatically retrieved and positioned in a recovery well.

Manual Operation

Whilst the operation of the above described apparatus is being described in an automatic process, it will be appreciated that the apparatus may be controlled manually. In order to achieve this, the processing system 10 is adapted to respond to input command provided by the user. The processing system 10 is adapted to respond to input commands to perform any one or more of:

Activate the laser;

Select marked cells from examination of images presented on the I/O device 22;

Control the positioning of the pipette; and,

Activating the pipette to recover one or more cells.

In this instance, the images of the inside of the selection well 71 can be displayed to the user on a suitable display, or the like. The user can then control the apparatus using suitable input commands to allow cells to be detected and recovered as described above, using the displayed images to determine the cell positions.

Cloning

The above described apparatus and method are particularly useful in the field of cloning, which refers to the isolation of a single cell into a vessel, plate, well etc. containing suitable growth media. In particular, the apparatus could be used as follows:
1. Single cell cloning based on visual identification by an operator. Using input commands and manual operation to isolate and recover single cells from within a culture, targeting cells by using the pipette system mounted on an inverted microscope.
2. Single cell cloning based on image analysis carried out by a computer. Using a camera mounted on an inverted microscope, live images can be captured and analysed by software, allowing the apparatus to target cells based on pre-set criteria such as shape, size and texture.
3. Single cell cloning based on fluorescent markers. Using either of the above methods but employing a fluorescent dye conjugated to a ligand, receptor, antibody or other molecule of interest. The dye could also be unconjugated. The dye could be stimulated by either a UV burner with suitable filters or a laser. Detection could be by either image capture or photodiode, either signal being interpreted by computer software and the pipette targeted to the cell of interest.
4. Multiple cell cloning. As above but with any number of cells as pre-set by the user.

In this instance, cells could be cloned from the following sources:
1. Non adherent cells contained in, and resting on the bottom of, a standard biological cell culture vessel.
2. As above but for adherent cells whereby a small dose (micro-injection) of suitable enzyme, typically a protease, could be administered using a multi-pipette to effect the release of cells from the surface of the vessel. Alternatively, suction alone might provide sufficient force in some instances to remove the adherent cells.
3. As above but for cells in a slice of tissue immersed in suitable media, whereby a small dose (micro-injection) of suitable enzyme could be administered using a multi-pipette to effect the release of cells from the tissue sample.

Accordingly, the system described above allows individual cells to be easily selected. As the cells are selected using the pipette as shown in FIG. 3, this makes individual cell selection easier than in the prior art. This therefore helps increase the speed and ease with which individual cells can be selected, recovered and used is subsequent procedures.

In addition to this, the apparatus as a whole is generally less complicated, thereby helping reduce the cost, as well as easing use of the apparatus to perform cell recovery. As a result, recovery using the system described above can generally be achieved more rapidly and cheaper than in the prior art

Accordingly, it will be appreciated that the apparatus is ideal for use in the following applications:
1. Rare Cell Recovery: Whereby there is a large number of cells are in culture and a small sub-population need to be recovered.
2. Diagnostics: Cell recovery from tissue obtained from a needle biopsy, along with the ability to clone single cell cultures of these cells and monitor subsequent growth and other characteristics such as surface marker expression.

Persons skilled in the art will appreciate that numerous variations and modifications will become apparent. All such variations and modifications which become apparent to persons skilled in the art, should be considered to fall within the spirit and scope that the invention broadly appearing before described.

Accordingly, while the above description has focused on cell selection, it will be appreciated that the techniques may generally be applied to any cells, vectors, particles, molecules, liposomes, and other such vesicles. Cells are defined as, but not limited to as being cells from vertebrate (including all mammalian species), invertebrate, plant, fungus and bacterial organisms, including all cells of eukaryotic and prokaryotic origin.

Furthermore, the techniques may be advantageously used in conjunction with the techniques described in the copending applications entitled “A Method of Cell Therapy” and “Cell Fusion”.

In particular, in this latter case, the automated selection and retrieval of cells advantageously allows single cell fusion to be performed at a high rate, allowing a large number of high quality fusates to be obtained.

1) Apparatus for recovering selected particles from a number of particles, the selected particles having predetermined properties, the apparatus including:
a) A stimulation system;
b) A detection system;
c) A retriever including;
i) A pipette;
ii) A drive system, coupled to the pipette;
iii) An actuator adapted to actuate the pipette to thereby expel or draw particles through a port; and,
d) A control system, coupled to the stimulation and detection systems and a pipette, the control system being adapted to:
i) Attempt to stimulate one or more of the particles using the stimulation system;
ii) Detect at least one stimulated particle using detection system, the stimulated particle having the predetermined properties;
iii) Operate the drive system to position port adjacent the stimulated particle; and,
iv) Recover the particle by operating the actuator to thereby draw the particle into the pipette.

2) Apparatus according to claim 1, the drive system being a micromanipulator.

3) Apparatus according to claim 1 or claim 2, the detection system being adapted to:
a) Determine the position of the stimulated particle; and,
b) Transfer an indication of the position of the stimulated to the control system, the control system being adapted to operate the drive system in accordance with the indicated stimulated particle position.

4) Apparatus according to any one of the claims 1 to 3, the stimulation system being coupled to the pipette, thereby allowing the stimulation system to stimulate particles near the port.

5) Apparatus according to claim 4, the control system being adapted to:
a) Operate the drive system until a number of particles are detected by the detection system;
b) Attempt to stimulate the particles; and,
c) Repeat steps (a) and (b) until one or more stimulated particles are detected.

6) Apparatus according to any one of the claims 1 to 5, the particles having the predetermined properties being coupled to respective markers, the stimulation system being adapted to stimulate the particles by stimulating the markers.

7) Apparatus according to claim 6, the markers being fluorescent markers, and the stimulation system including a radiation source for stimulating the fluorescent markers.

8) Apparatus according to claim 7, the radiation source being a laser.

9) Apparatus according to claim 6, the markers being magnetic markers, the stimulation system being formed from a magnetic field generator adapted to generate a magnetic field.

10) Apparatus according to claim 9, the magnetic field being adapted to attract stimulated particles towards the retriever for recovery.

11) Apparatus according to claim 10, the stimulated particles being drawn into the pipette under the action of the magnetic field.

12) Apparatus according to any one of the claims 9 to 11, the detection system being formed from the magnetic field generator, the stimulated particles being determined by the attraction of the particles to the magnetic field.

13) Apparatus according to any one of the claims 1 to 12, the detection system including:
a) An imaging system for generating images of one or more of the particles; and,
b) A processing system for detecting stimulated particles in the images.

14) Apparatus according to claim 1 and claim 13, the imaging system being coupled to the drive system to thereby generate images of a region near the port.

15) Apparatus according to any one of the claims 1 to 14, the particles being cells.

16) Apparatus for recovering selected particles from a number of particles, the selected particles having predetermined properties, the apparatus being substantially as hereinbefore described.

17) A method of recovering selected particles from a number of particles, the selected particles having predetermined properties, the method including:
a) Attempt to stimulate one or more of the particles;
b) Detect at least one stimulated particle having the predetermined properties; and,
c) Recover a stimulated particle using a retriever the retriever including a pipette, a drive system, and actuator adapted to actuate the pipette to thereby expel or draw in particles through a port, the method including:
i) Operating the drive system to position port adjacent the stimulated particle; and,
ii) Recovering the particle by operating the actuator to thereby draw the particle into the pipette.

18) A method according to claim 17, the method including using i control system to operate the drive system and the actuator.

19) A method according to claim 17 or claim 18 the method including using a detection system to:
a) Determine the position of the stimulated particle; and,
b) Operate the drive system in accordance with the indicated stimulated particle position.

20) A method according to claim 19 the method including:
a) Using an imaging system for generating images of one or more of the particles; and,
b) Using a processing system for detecting stimulated particles in the images.

21) A method according to claim 19, the method including causing the control system to:
a) Operate the drive system until a number of particles are detected by the imaging system;
b) Attempt to stimulate the particles; and,
c) Repeat steps (a) and (b) until one or more stimulated particles are detected.

22) A method according to any one of the claims 17 to 21, the method including:
a) Coupling the particles having the predetermined properties to respective markers; and,
b) Stimulating the particles by stimulating the markers.

23) A method according to claim 22, the markers being fluorescent markers, and the method including stimulating the markers using a radiation source.

24) A method according to claim 22, the markers being magnetic markers, the method including applying a magnetic field to the particles to thereby stimulate the particles.

25) A method according to claim 24, the magnetic field being adapted to attract stimulated particles towards the retriever for recovery.

26) A method according to claim 25, the stimulated particles being drawn into the pipette under the action of the magnetic field.

27) A method according to any one of the claims 24 to 26, the method including detecting stimulation of the particles by detecting the attraction of the particles towards the retriever.

28) A method according to any one of the claims 17 to 27, the particles being cells.

29) A method according to any one of the claims 17 to 28, the method being performed by apparatus according to any one of the claims 1 to 16.

30) A method for recovering selected particles from a number of particles, the selected particles having predetermined properties, the method being substantially as hereinbefore described.

31) A processing system for controlling apparatus for recovering selected particles from a number of particles, the selected particles having predetermined properties, the apparatus including a stimulation system, a detection system and a retriever, the processing system being adapted to:
a) Attempt to stimulate one or more of the particles using the stimulation system;
b) Detect at least one stimulated particle using detection system, the stimulated particle having the predetermined properties; and,
c) Recover a stimulated particle using the retriever, the retriever including a pipette, a drive system, coupled to the pipette and an actuator adapted to actuate the pipette to thereby expel or draw particles through a port, by:
i) Operating the drive system to a position port adjacent the stimulated particle; and,
ii) Recovering the particles by operating the actuator to thereby draw the particle into the pipette.

32) A processing system according to claim 31, the processing system being adapted to perform the method of any one of the claims 17 to 29.

33) A processing system according to claim 31 or claim 32, the processing system being adapted to operate as the control system of any one of the claims 1 to 16.

34) A processing system for controlling apparatus for recovering selected particles from a number of particles, the selected particles having predetermined properties, the apparatus including a stimulation system, a detection system and a retriever, the processing system being substantially as hereinbefore described.

35) A computer program product apparatus for recovering selected particles from a number of particles, the selected particles having predetermined properties, the apparatus including a stimulation system, a detection system and a retriever, the computer program product including computer executable code which when executed by a suitable processing system causes the processing system to perform the method of any of the claims 17 to 29.

36) A computer program product apparatus for recovering selected particles from a number of particles, the selected particles having predetermined properties, the apparatus including a stimulation system, a detection system and a retriever, the computer program product including computer executable code which when executed by a suitable processing system causes the processing system to operate substantially as hereinbefore described.

37) A pipette system for manipulating particles, the pipette system including:
a) A pipette for containing fluid in use, the pipette including a port;
b) An actuator coupled to the pipette, the actuator being adapted to draw in and/or expel fluid through the port;
c) A radiation source; and,
d) A waveguide having a first end coupled to the radiation source and a second end coupled to the pipette adjacent the port to thereby allow radiation from the radiation source to impinge on particles positioned adjacent to the port in use.

38) A pipette system according to claim 37, the pipette system including a detector, the detector being adapted to detect radiation emitted by the particle.

39) A pipette system according to claim 38, the detector being coupled to the first end of the waveguide, to thereby detect radiation emitted from the particle.

40) A pipette system according to any one of the claims 37 to 39, the radiation source being a laser.

41) A pipette system according to any one of the claims 37 to 40, the waveguide being a fibre optic cable.

42) A pipette system according to any one of the claims 37 to 41, the waveguide being formed from the pipette, the pipette including a shaped portion to allow the radiation from the radiation source to enter the pipette and pass along at least a portion of the pipette, the radiation being emitted from the pipette through the port

43) A pipette system according to any one of the claims 37 to 42, the pipette system including a controller adapted to perform at least one of:
a) Activating the actuator to thereby cause fluid to be drawn in and/or expelled through the port; and,
b) Activating the radiation source, to thereby expose a particle to radiation.

44) A pipette system according to any one of the claims 37 to 43, the pipette system including a drive system adapted to move the pipette system to be with respect to a fluid filled container to thereby allow particles to be positioned in or removed from fluid in the container.

45) A pipette system according to claim 44, the drive system being coupled to a controller, the controller being adapted to recover particles having predetermined properties from the container by:
a) Positioning the pipette system such that the port is adjacent to a particle;
b) Activating the radiation source to thereby expose the particle to radiation;
c) Detect any radiation emitted by the particle;
d) Determine if the particle has the predetermined properties in accordance with the detected radiation; and,
e) In accordance with a successful comparison, activate the actuator to thereby draw fluid into the pipette through the port, thereby recovering the particle.

46) A pipette system according to claim 43 or claim 45, the controller being formed from a suitably programmed processing system.

47) A pipette system for manipulating particles, the pipette system including:
a) A pipette for containing fluid in use, the pipette including a port;
b) An actuator coupled to the pipette, the actuator being adapted to draw in and expel fluid through the port, the actuator including:
i) A fluid reservoir,
ii) A flexible tube coupling the pipette to the fluid reservoir;
iii) An arm positioned so as to partially compress the tube;
iv) An actuator drive system adapted to move the arm so as to perform at least one of:
(1) Further compressing the tube to thereby expel fluid from the port, and,
(2) Decompressing the tube to thereby draw fluid in through the port.

48) A pipette system according to claim 47, the actuator drive system including:
a) A first actuator drive for moving the arm with respect to the tube; and,
b) A second actuator drive formed from an arm end portion, the arm end portion being in contact with the tube in use, the second actuator drive being adapted to cause the arm end portion to expand or contract.

49) A pipette system according to claim 47 or claim 48, the pipette system including a controller coupled to the actuator drive system, the controller being adapted to operate the actuator drive system to thereby draw fluid in or expel fluid through the port

50) A pipette system according to any one of the claims 47 to 49, the pipette system including a drive system adapted to move the pipette system to be with respect to a fluid filled container to thereby allow particles to be positioned in or removed from fluid in the container.

51) A pipette system according to claim 50, the drive system being coupled to the controller, the controller being adapted to recover particles from the fluid by:
a) Positioning the pipette system such that the port is adjacent to a particle; and,
b) Activate the actuator drive system to thereby draw fluid into the pipette through the port, thereby recovering the particle.

52) A pipette system according to any one of the claims 47 to 51, the tube being formed from silicon tubing.

53) A pipette system according to any one of the claims 47 to 52, the pipette system being a pipette system according to any of the claims for manipulating system 37 to 46.

54) A pipette system for manipulating particles, the pipette system being a pipette system substantially as hereinbefore described with reference to the accompanying drawings.

55) A pipette system for manipulating particles, the pipette system including:
a) A pipette for containing fluid in use, the pipette including a port;
b) An actuator coupled to the pipette, the actuator being adapted to draw in and/or expel fluid through the port; and,
c) A magnetic field generator for exposing particles to a magnetic field.

56) A pipette system according to claim 55, the magnetic field generator being formed from a solenoid.

57) A pipette system according to claim 56, the pipette including a nozzle, the solenoid being formed from a graphite layer and a number of associated windings arranged on the nozzle.

58) A pipette system according to claim 57, the pipette system including a controller adapted to perform at least one of:
a) Activating the actuator to thereby cause fluid to be drawn in and/or expelled through the port, and,
b) Activating a current supply coupled to the solenoid, to thereby expose the particles to a magnetic field.

59) A pipette system according to any one of the claims 55 to 58, the pipette system including a drive system adapted to move the pipette system to be with respect to a fluid filled container to thereby allow particles to be positioned in or removed from fluid in the container.
